(19) United States
(12) Patent Application Publication
(10) Pub. No.: US 2023/0271107 A1
(43) Pub. Date: Aug. 31, 2023

(54) APPARATUS FOR EXTRACTING PLANT COMPOUNDS AND RELATED METHOD

(71) Applicant: Angstead Custom Engineering, LLC, Denver (US)

(72) Inventors: Ryan P. Angstead, Aurora, CO (US); Andrew Trainer, Westminster, CO (US)

(73) Assignee: Angstead Custom Engineering, LLC, Denver (US)

(21) Appl. No.: 18/174,615

(22) Filed: Feb. 25, 2023

Related U.S. Application Data
(60) Provisional application No. 63/314,393, filed on Feb. 26, 2022.

(51) Int. Cl. B01D 11/02; B01D 8/00; A61K 36/185

(52) U.S. Cl. CPC B01D 11/0296; B01D 11/0288; B01D 11/0207; B01D 8/00; A61K 36/185; A61K 2236/37; A61K 2236/51; A61K 2236/35

(57) ABSTRACT

Extraction devices and related methods are disclosed. Extraction devices disclosed may include a hopper, a piston, and an evaporation chamber. The hopper may be filled with a plant material and a solvent. The piston may be removably housed within the hopper so as to form a hermetic seal with the hopper. Once a solvent and extract mixture is formed within the hopper, a valve in the bottom of the hopper may be opened to allow the solvent and extract mixture to flow into the evaporation chamber via gravity. The remaining plant material may be compressed by the piston to remove any remaining solvent and extract mixture. The solvent may be evaporated in the evaporation chamber leaving a purified plant extract. Various types of plant materials may be used within the hopper. Various types of solvents may be used as well, including heptane and hexane.

### RELATED APPLICATIONS

This application claims priority to U.S. Provisional Application Ser. No. 63/314,393, filed Feb. 26, 2022.

### BACKGROUND OF THE INVENTION

Extracting compounds from plants is well known in the art. Compounds extracted from herbs and other plant materials have many known and beneficial uses. Plant extracts have been used in cosmetics, for pigments, for medicinal purposes, as additives to food, perfumes, flavorings, poisons, etc. For example, compound extraction from the Cannabis plant has traditionally been used as a mild analgesic. Cannabis extract has also been shown to give relief to individuals suffering from asthma, arthritis, migraines, chronic pain, multiple sclerosis, glaucoma, epilepsy, to chemotherapy patients to help with nausea and vomiting, among other conditions. Generally, the purer the oil extract from a plant, the better it is for any given purpose.

Accordingly, there has been an ongoing need for improvements in methods and apparatuses for the extraction of plant compounds. Therefore, new methods and apparatuses for the extraction of plant compounds would be well received by the general public.

### SUMMARY OF THE INVENTION

An extraction device may have a reservoir configured to hold a solvent, a hopper configured to receive a plant material and to receive the solvent from the reservoir so as to form a plant material and solvent mixture within the hopper, a piston configured to be removably housed within the hopper so as to form a hermetic seal when housed therein and configured to compress the plant material and solvent mixture to force a solvent and extract mixture from the hopper, a chamber configured to receive the solvent and extract mixture from the hopper and to evaporate the solvent from the solvent and extract mixture leaving a purified plant extract mixture within the chamber, a condenser configured to receive the evaporated solvent from the chamber and to condense the evaporated solvent so that the condensed solvent may be returned to the reservoir.

The reservoir may be hermetically connected to the hopper through a reservoir-hopper link having a first end connected to the reservoir and a second end connected to the hopper so as to form a fluid passageway from the reservoir to the hopper. The solvent from the reservoir may be introduced into the hopper through the reservoir-hopper link by means of a solvent pump.

The hopper may be hermetically connected to the chamber through a hopper-chamber link having a first end connected to the hopper and a second end connected to the chamber so as to form a fluid passageway from the hopper to the reservoir. Mixing solvent with the plant material in the hopper may create a solvent and extract mixture within the hopper. The hopper may include a filter or mesh proximate to the first end of the hopper-chamber link with a valve between the filter or mesh and the first end of the hopper-chamber link such that when the valve is opened the solvent and extract mixture may flow from the hopper to the chamber through the hopper-chamber link by means of gravity. The piston may further compress the plant material against the filter or mesh so as to squeeze any remaining solvent and extract material from the plant material so that it flows through the hopper-chamber link and into the chamber.

The chamber may be hermetically connected to the condenser through a chamber-condenser link having a first end connected to the chamber and a second end connected to the condenser so as to allow the evaporated solvent from the chamber to travel from the chamber through the chamber-condenser link and into the condenser.

The condenser may be hermetically connected to the reservoir through a condenser-reservoir link having a first end connected to the condenser and a second end connected to the reservoir so as to allow the condensed solvent from the condenser to flow from the condenser, through the condenser-reservoir link, and into the reservoir. The condenser may have a sight glass so as a user may observe the drip and flow of the condensed solvent from the condenser and into the condenser-reservoir link. The condenser may further have a light so as to illuminate the sight glass making observation of the drip and flow of the condensed solvent easier.

The extraction device may also have a control panel configured to dictate the operation of the extraction apparatus. The control panel may be in electrical communication with one or more of the various components of the extraction device.

The extraction device may also have an ice trap. The ice trap may be hermetically connected between the first and second ends of the condenser-reservoir link so as to capture and condense any remaining evaporated solvent that may have passed through the condenser without condensing. In some embodiments, the ice trap may be closer to the second end of the condenser-reservoir link than to the first end.

The extraction device may also have a coolant subsystem comprised of a coolant reservoir, a coolant pump, and coolant line. The coolant reservoir may be configured to hold a coolant, the coolant pump may be configured to pump the coolant from the coolant reservoir and through the coolant line, and the coolant line may be configured to wrap around, pass through, or otherwise be proximate to the hopper and condenser so as to function to cool the hopper and the condenser.

In some embodiments, the coolant line may bifurcate into two lines wherein the first line wraps around, passes through, or is otherwise proximate to the hopper and wherein the second line wraps around, passes through, or is otherwise proximate to the condenser. Valves may be placed in the first line and in the second line. These valves may be turned on and off allowing or preventing coolant from flowing to the hopper and the condenser.

The coolant subsystem may also be comprised of a thermal mass chiller. The coolant line may pass through the thermal mass chiller before returning to the coolant reservoir. The thermal mass chiller may function to prechill the returning coolant before it enters the coolant reservoir.

The extraction device may have a freezer. The freezer may house one or more components of the extraction device. The solvent reservoir may be housed in the freezer along with the ice trap. The coolant reservoir may also be housed in the freezer along with the thermal mass chiller. The solvent pump and the coolant pump may also be housed in the freezer.

As partially described above, the extraction device may have one or more sight glasses configured to allow a user to observe the functioning of one or more components of the extraction device. For example, the condenser may have a sight glass to observe the functioning of the condenser, the hopper may have a sight glass to observe the functioning of the hopper, the chamber may have a sight glass to observe the functioning of the chamber, etc. One or more lights may be associated with any of the one or more sight glasses so as to illuminate the sight glasses for better observation by a user.

The solvent reservoir may be configured to hold heptane or hexane as a solvent. The extraction device may also be configured to be optimized for use the use of heptane or hexane as a solvent.

In one embodiment, the extraction device may be divided into one or more subsystems which may include a solvent subsystem, a piston subsystem, an evaporation subsystem, a condenser subsystem, a coolant subsystem, and a vacuum subsystem. One or more of the subsystems may be in electrical communication with a control panel in which the control panel may dictate the function of the one or more subsystems.

The solvent subsystem may be comprised of a solvent reservoir, a solvent pump and solvent lines. The solvent reservoir may hold a solvent such as heptane or hexane. The solvent pump may pump solvent from the solvent reservoir and into the solvent lines. The solvent lines may allow solvent to be introduced to the piston subsystem. The solvent lines may also allow solvent to pass from the evaporation subsystem to the condenser subsystem. The solvent lines may also allow solvent to be collected from the condenser subsystem and returned to the solvent reservoir.

The piston subsystem may be comprised of a piston and a hopper. The hopper may be configured to receive plant material and solvent. The piston may be configured to be removably housed within the hopper and to compress the plant material and solvent mixture so as to push a solvent and extract mixture from the hopper and into the evaporation subsystem.

The evaporation subsystem may be comprised of a chamber, a heat element and a stir rod. The chamber may receive the solvent and extract mixture. The heat element may heat up the solvent and extract mixture so as to evaporate the solvent from the solvent and extract mixture. The stir rod may function to stir the solvent and extract mixture.

The condenser subsystem may be comprised of a condenser and an ice trap. The condenser may function to collect evaporated solvent from the evaporation subsystem and to condense the evaporated solvent back to a liquid. The ice trap may function to capture any remaining evaporated solvent that made it past the condenser and to condense the remaining evaporated solvent into a liquid.

The coolant subsystem may be comprised of a coolant reservoir, a coolant pump, coolant lines, and a thermal mass chiller. The coolant reservoir may hold a coolant. The coolant pump may pump coolant from the coolant reservoir. The coolant lines may circulate coolant through, around or otherwise proximate to the hopper and the condenser so as to cool the hopper and the condenser. The coolant lines may also run through a thermal mass chiller so as to prechill the coolant before it reenters the coolant reservoir through the coolant lines.

The vacuum subsystem may be comprised of one or more vacuums, a vacuum regulator, and vacuum lines. The vacuums may lower the pressure in the evaporation subsystem and may also function to assist in pulling solvent and extract mixture from the hopper and into the evaporation subsystem.

The extraction device may further have a freezer and a frame. The freezer my hold some of the components of the extraction device such as, but not limited to, the solvent reservoir, the solvent pump, the ice trap, the coolant reservoir, the coolant pump, and the thermal mass chiller. The freezer may function to keep the components held within cool. The frame may function to support the various subsystems and components of the extraction device.

A method of extracting a plant extract is also disclosed. The method may contain the step of filling a hopper with a plant material and a solvent. The solvent may be heptane or hexane. Another step may be allowing the plant material and solvent to soak until a solvent and extract mixture is formed. Another step may be separating the solvent and extract mixture from the plant material and into an evaporation chamber using gravity. Another step may be compressing the plant material with a piston within the hopper to further separate any remaining solvent and extract mixture from the plant material and into the evaporation chamber. Another step may be repeating the above listed steps one or more times. Another step may be heating the solvent and extract mixture in the evaporation chamber to separate the solvent from the extract through evaporation of the solvent. Another step may include condensing the evaporated solvent and reclaiming the solvent into a solvent reservoir. Another step may include collecting the separated extract from the evaporation chamber.

Other features and advantages of the various embodiments of the extraction device and associated method of extracting plant extract will become apparent from the following more detailed description, taken in conjunction with the accompanying drawings, which illustrate, by way of example, the principles of the various embodiments of the extraction device and associated method.

### BRIEF DESCRIPTION OF THE DRAWINGS

Various embodiments will be described and explained with additional specificity and detail through the use of the accompanying drawings in which:

FIG. 1 illustrates an exploded perspective view of a frame, a control panel, and various subsystems of an extraction apparatus;

FIG. 2 illustrates a front perspective view of the extraction apparatus of FIG. 1;

FIG. 3 illustrates an evaporation chamber side perspective view of the extraction apparatus of FIGS. 1 and 2;

FIG. 4 illustrates a freezer side perspective view of the extraction apparatus of FIGS. 1-3;

FIG. 5 illustrates a perspective view of a control panel of the extraction apparatus of FIGS. 1-3;

FIG. 6 illustrates a perspective view of a plant hopper, a condenser, and coolant lines of the extraction apparatus of FIGS. 1-3;

FIG. 7 illustrates a perspective view of a piston drive system in an up position of the extraction apparatus of FIGS. 1-3;

FIG. 8 illustrates a perspective view of the piston drive system in a down position of the extraction apparatus of FIGS. 1-3;

FIG. 9 illustrates a perspective view of a piston and piston housing of the piston drive system illustrated in FIGS. 7 and 8;

FIG. 10 illustrates a perspective view of an evaporation chamber of the extraction apparatus of FIGS. 1-3;

FIG. 11 illustrates an exploded perspective view of the components contained within the freezer of the extraction apparatus of FIGS. 1-3;

FIG. 12 illustrates a perspective view of components contained within the freezer of the extraction apparatus of FIGS. 1-3;

FIG. 13 illustrates a perspective view of coolant, solvent, and vacuum lines extending into the freezer of the extraction apparatus of FIGS. 1-3,

FIG. 14 illustrates a perspective view of coolant, solvent, and vacuum lines extending out of the freezer of the extraction apparatus of FIGS. 1-3; and

FIG. 14 illustrates a flowchart of steps in a method of extracting plant compounds.

### DETAILED DESCRIPTION OF THE PREFERRED EMBODIMENTS

As shown in the drawings and for purposes of illustration, the one or more embodiments disclosed herein illustrate an extraction apparatus generally referred to herein by the reference numeral 100 and a related method of extracting plant compounds. Generally, the extraction apparatus 100 may extract, purify, and/or concentrate oils and compounds from plant material that may be loaded into the extraction apparatus 100. Various types of plant material may be loaded into the extraction apparatus 100 resulting in various types of oils and compounds being extracted, purified, and/or concentrated. One example application of the extraction apparatus is to extract, purify, and/or concentrate hash oil from the cannabis plant through the use of heptane or hexane as a solvent. Generally, the extraction apparatus 100 may be comprised of a frame 102, a control panel 200 and one or more subsystems. The frame 102 may hold in place and support the control panel 200 and the one or more subsystems. The control panel 200 may act as the “brains” of the extraction apparatus 100 and may dictate and control the operations, interworking, and coordination of the one or more subsystems of the extraction apparatus 100.

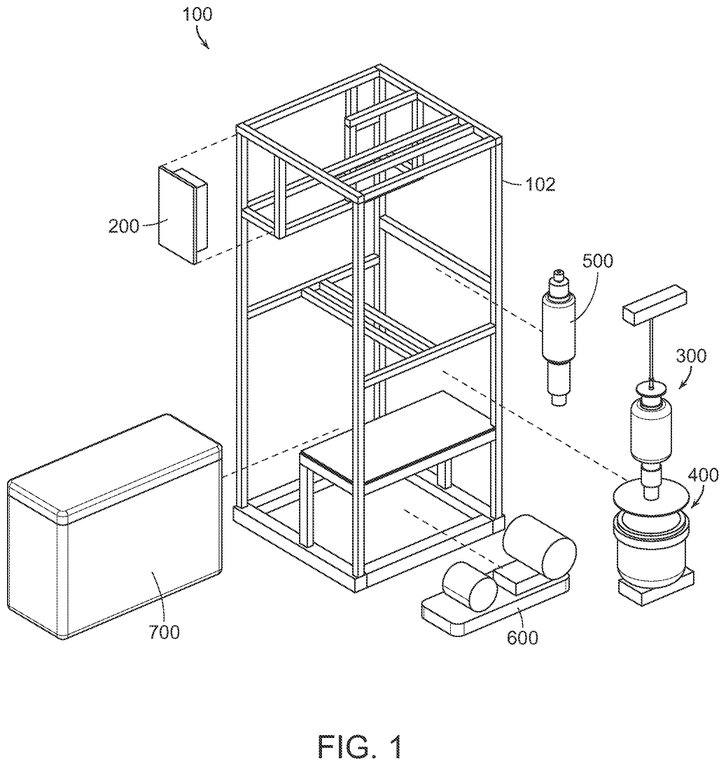

With reference now to FIG. 1, an exploded perspective view illustrates various parts and subsystems of the extraction apparatus 100. As previously described above, the extraction apparatus 100 may be comprised of a frame 102 which holds and supports the control panel 200 and one or more subsystems. The extraction apparatus 100 may be comprised of a piston subsystem 300, an evaporation chamber subsystem 400, a condenser subsystem 500, a vacuum subsystem 600, and freezer components 700 which may hold components of a solvent subsystem, a coolant subsystem, the condenser subsystem, and the vacuum subsystem. One or more of the subsystems may be interconnected to form, in the totality, a closed system. The one or more subsystems may also be functionally connected to the control panel 200.

The frame 102 of the extraction apparatus 100, as previously described above, may function to hold and support the control panel 200 and the one or more subsystems in their correct positions in relation to each other. The frame 102 may be cuboidal in shape and may be comprised of one or more horizontal bars, one or more vertical bars, and one or more platforms. It would be recognized by one skilled in the art that the bars of the frame 102 may be made from any strong and sturdy material that would hold and support the control panel 200, one or more subsystems, and the various electrical, fluid, and other connections of the extraction apparatus 100. In a preferred embodiment, channeled interconnecting metal bars which are capable of easily securing clamps and other components and which are capable of connecting together without welding may be used as the bars of the frame 102. Generally, the frame 102 may have a section to support the control panel 200 and various electronic components of the extraction apparatus 100, bars to support the condenser subsystem 500, bars to support the piston subsystem 300, a section to support the evaporation chamber subsystem 400, a section to support the vacuum subsystem 600, and a section to support the freezer components 700. Generally, the condenser subsystem 500 and the piston subsystem 300 may be superiorly positioned to the evaporation chamber subsystem 400 and the evaporation chamber subsystem 400 may be superiorly positioned to the freezer components 700. The control panel 200 may be positioned in an upper corner of the frame 102 so as to allow easy access to a user of the extraction apparatus 100. The vacuum subsystem 600 may be positioned inferiorly to the evaporation chamber subsystem 400.

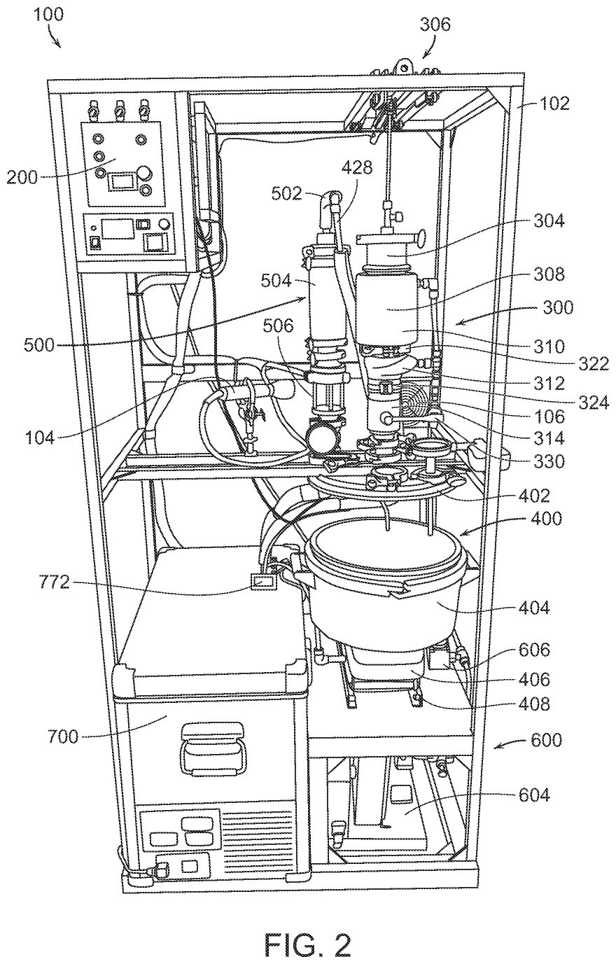

With reference now to FIGS. 2-4, various perspective views of the extraction apparatus 100, as a whole, are illustrated. FIGS. 2-4 illustrate a preferred placement of the control panel 200 within the frame 102. Further, FIGS. 2-4 illustrate the preferred placement of the various subsystems, which may include the condenser subsystem 500, the piston subsystem 300, the evaporation chamber subsystem 400, the vacuum subsystem 600, and the freezer components 700 as already described above. In addition, FIGS. 2-4 illustrate the various coolant lines, electrical lines, and interconnectability of the various subsystems. Finally, FIGS. 2-4 illustrate additional subcomponents of the extraction apparatus 100. For example, the extraction apparatus 100 may include lighting 104 to help a user monitor the functioning of the extraction apparatus 100. Furthermore, the extraction apparatus 100 may include a fan 106 to help regulate the temperature of the extraction apparatus 100 during operation. FIGS. 2-4 will be frequently referred to throughout the following disclosure.

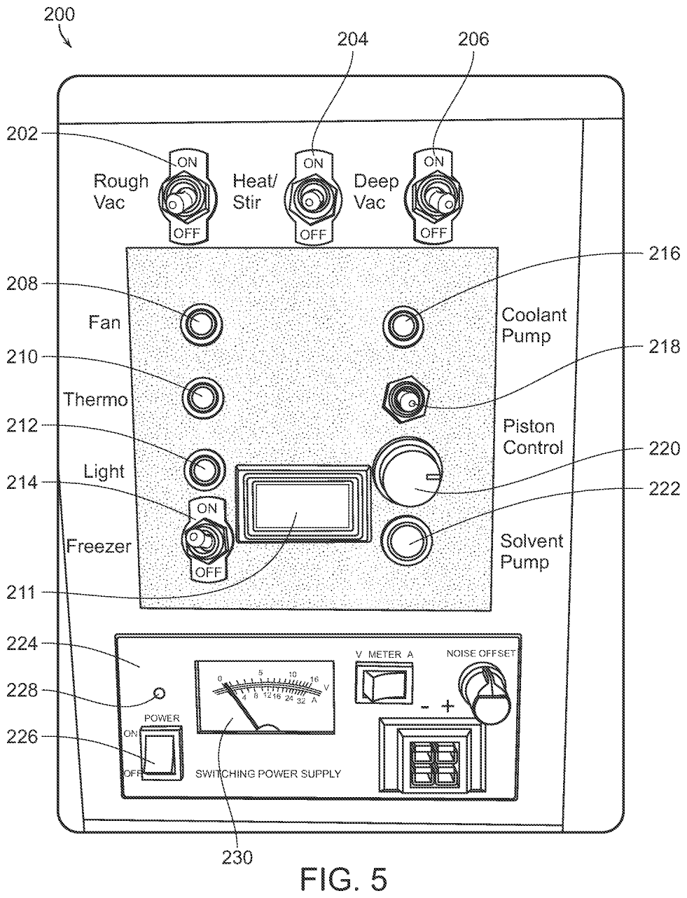

With reference now to FIG. 5, the control panel 200 of the extraction apparatus 100 is illustrated. As described above, the control panel 200 may act as the “brains” of the extraction apparatus 100. The control panel 200 may control the operation of the extraction apparatus 100 as a whole and may further control the operations of the various subsystems described above and which will be described in further detail below. The control panel 200 may be comprised of one or more of the following circuit controls: a first vacuum switch 202, a heat/stir switch 204, a second vacuum switch 206, a fan switch 208, a thermometer switch 210, a thermometer display 211, a light switch 212, a freezer switch 214, a coolant pump switch 216, a piston up/down switch 218, a piston speed switch 220, and a solvent pump switch 222. The term “switch”, as used above, may mean any of the following: a switch, a button, a knob, a touch sensor, or any other device that interrupts or alters the electron flow in a circuit known in the art. Each switch may be operably connected to a circuit providing power to the system or part for which the switch is named. For example, the first vacuum switch 202 may be operably connected to the circuit providing power to a first vacuum, the heat/stir switch 204 may be operably connected to the circuit providing power to a heat/stir source, and so forth.

The control panel 200 may also be comprised of a first power supply 224. The first power supply 224 may supply power to one or more of the circuits of the circuit controls described above. The first power supply 224 may be comprised of an on/off switch 226, a power indicator 228, and a voltage meter 230. The first power supply 224 may be a twelve-volt power supply and may preferably supply power to the circuits of the fan switch 208, the thermometer switch 210, the light switch 212, the freezer switch 214, the coolant pump switch 216, the piston up/down switch 218, the piston speed switch 220, and the solvent pump switch 222.

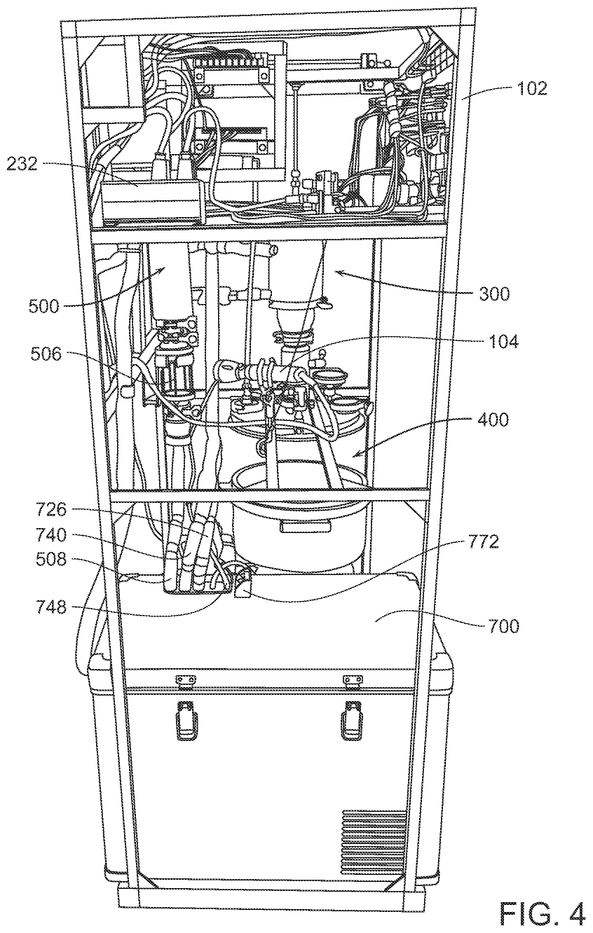

A second power supply 232, as best seen in FIG. 4, may also be provided. The second power supply 232 may also supply power to one or more of the circuits of the circuit controls described above. The second power supply 232 may be a one-hundred-and-ten-volt power supply and may preferably supply power to the circuits of the first vacuum switch 202, the heat/stir switch 204, and the second vacuum switch 206. Both the first power supply 224 and the second power supply 232 may receive power from an outside power source such as from any known electrical outlet known in the art. In the alternative, the first power supply 224 may receive power internally from the second power supply 232 via an electrical connection and the second power supply 232 may receive power externally from an outside power source such as from any electrical outlet known in the art.

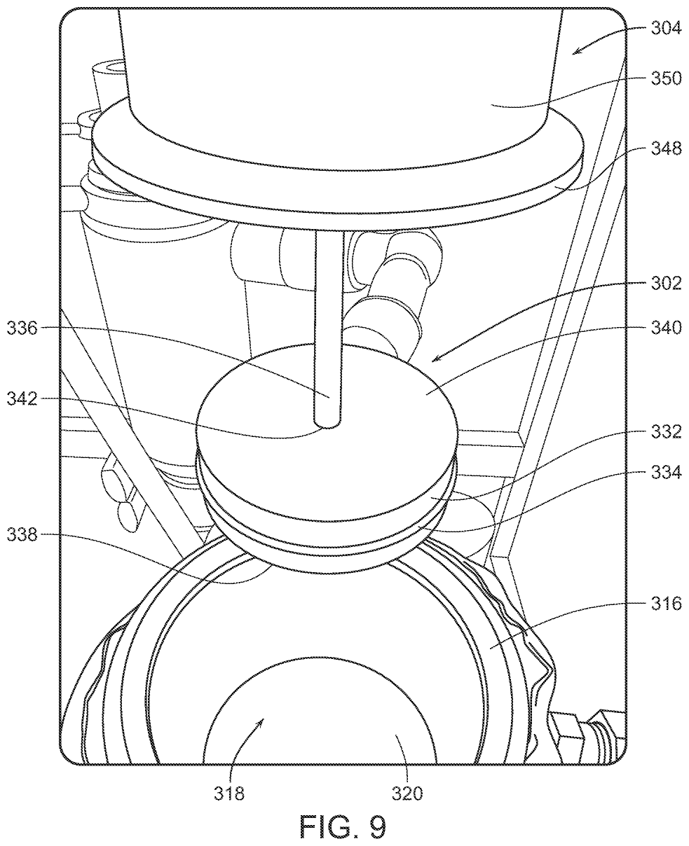

With reference now to FIGS. 2, 3 and 6-10, the piston subsystem 300 is best shown. Generally, the piston subsystem 300 may function to soak plant material in a solvent, regulate the temperature of the plant material and solvent mixture, drain solvent away from the plant material, and press remaining solvent out of the plant material. The piston subsystem 300 may generally be comprised of a piston 302, a piston housing 304, a piston drive system 306, and a plant hopper 308.

The plant hopper 308 may be configured to hold a plant material and to have a solvent introduced to mix with the plant material. The plant hopper 308 may be comprised of three main segments which may include an upper body 310, a lower body 312 and a valve 314. These three segments may be one solid piece or may be three distinct segments that are hermetically sealed together. The upper body 310 may have a first end comprised of a rim 316 defining an opening to a compartment 318 of the upper body 310, a second end 320 comprised of a grate, and a height separating the first end from the second end 320. The lower body 312 may similarly have a first end 322, a second end 324, and a height separating the first end 322 from the second end 324. The lower body 312 may also have an inner compartment that is fluidly connected to the compartment 318 of the upper body 310. The lower body 312 may be funneled in shape such that the diameter of the first end 322 may be larger than the diameter of the second end 324. Alternatively, the upper body 310 and the lower body 312 may be tubular in shape having with the grate separating the upper body 310 from the lower body 312 and the diameter of horizontal cross-sections of the inner surface of the upper body 310 and the lower body 312 being approximately of equal diameter.

As described above, the grate of the upper body 310 may separate the compartment 318 of the upper body 310 from the inner compartment of the lower body 312. The grate may allow liquid to pass through from the compartment 318 of the upper body 310 to the inner compartment of the lower body 312 but may not allow larger material such as plant material to pass from the compartment 318 of the upper body 310 to the inner compartment of the lower body 312. The first end 322 of the lower body 312 may be one with the second end 320 of the upper body 310. Alternatively, the first end 322 of the lower body 312 may be hermetically sealed to the second end 320 of the upper body 310.

The valve 314 may have a top 326, a bottom 328, and a handle 330. The top 326 may be one with the second end 324 of the lower body 312. Alternatively, the top 324 of the valve 314 may be hermetically sealed to the second end 324 of the lower body. The handle 330 of the valve 314 may cause the valve 314 to open and close when turned. The valve may be a ball valve, a butterfly valve, a gate valve, or any other type of valve known in the art. The valve 314 may act to keep solvent and any solute from the plant material within the plant hopper 308. When an operator uses the handle 330 to open the valve 314, the solvent and any solute from the plant material may exit the plant hopper 308.

The upper body 310 of the plant hopper 308 may further be comprised of a coolant coil which spirals tightly around an exterior of the upper body 310. The coolant coil may allow coolant to circulate through and act to cool and regulate the temperature of the interior of the plant hopper 308, i.e., the compartment 318 of the upper body 310 and the compartment of the lower body 312. The upper body 310 may further be comprised of insulation around the coolant coil. The insulation may be wrapped tightly around and secured to the coolant coil and exterior of the upper body 310 to assist in regulating the temperature of the interior of the plant hopper 308. The insulation may similarly further extend around the lower body 312 so as to completely wrap around the exterior of the plant hopper 308.

The plant hopper 308 may further include a sight glass 309. The sight glass 309 may function to allow a user to determine the level of solvent within the plant hopper 308. The sight glass 309 may begin where a solvent fill line (discussed further below) enters into the plant hopper 308 and end near the top of the plant hopper 308. In some embodiments, the sight glass 309 may be a transparent tube, such as a Teflon tube that extends along the outside of the plant hopper 308. When solvent is introduced into the plant hopper, it will also be introduced into the sight glass 309 such that the level of the solvent in the sight glass 309 may indicate the level of the solvent in the plant hopper 308.

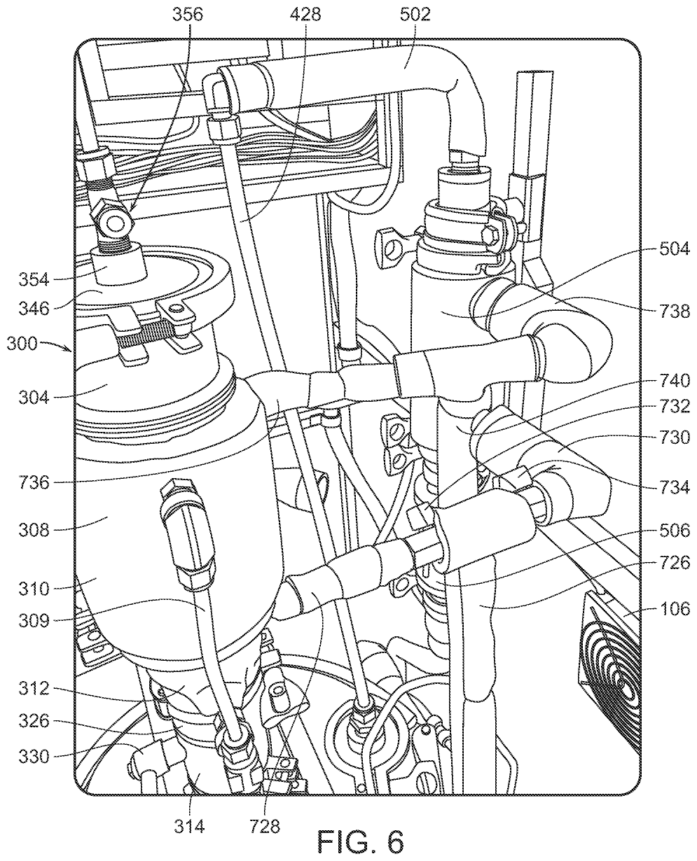

With reference now specifically to FIGS. 6-9, the piston 302, the piston housing 304, and the piston drive system 306 are best illustrated. Generally, the piston 302 may function, through assistance of the piston drive system 306, to compress the plant material, solvent, and solute mixture in the plant hopper 308 against the grate, when the valve 314 is in the open position, to further separate the solvent and solute from the plant material. The piston housing 304 may function to create a hermetically sealed system between the piston housing 304 and the plant hopper 308 with the piston inside of the piston housing 304 and/or the plant hopper 308.

The piston 302 may be comprised of a head 332, an O-ring 334, and a rod 336. The head 332 may be cylindrical in shape having a first face 338, a second face 340, and a height between the first face 338 and the second face 340. The piston 302 may be solid stainless steel or other similar material. The O-ring 334 may stretch tightly around the piston 302 between the first face 338 and the second face 340. The O-ring 334 may be comprised of a durable rubber material such as Teflon or other similar material known in the art. The piston 302 may further be comprised of a shallow channel in which the O-ring 334 may be secured and extend from to extend slightly past the exterior surface of the piston 302. The channel and O-ring may be located approximately in the middle of the piston between the first face 338 and the second face 340.

The rod 336 may be comprised of a first end 342, a second end 344, and a length between the first end 342 and the second end 344. The first end 342 of the rod may be permanently attached to or one with the head 332 of the piston 302. The first end 342 may be orthogonally attached to the center of the second face 340 of the head 332. The rod 336 may extend through an aperture in a top face 346 of the piston housing 304. The second end 344 of the rod 336 may be attached to the piston drive system 306 such that when the piston drive system 306 is actuated, the piston 302 moves up and down inside the plant hopper 208 and/or the piston housing 304.

The piston housing 304 may be cup-like in shape and may have a flat-lipped rim 348, a cylindrical wall 350, and a top face 352. The cylindrical wall 350 and the top face 352 may define a compartment within the piston housing 304 and the flat-lipped rim 348 may define an opening to the compartment of the piston housing 304. The compartment may house the piston head 332. The top face 352 of the piston housing 304 may have a cylindrical protuberance 354 defining an opening through which the piston rod 336 may extend through the top face 352 of the piston housing 304. The piston housing may further include means 356 for hermetically sealing the rod 336 and piston housing 304 as the rod 336 extends through the top face 352 of the piston housing 334.

The piston housing 304 may be hermetically sealed to the plant hopper 308. For example, with the piston within the piston housing 304, a gasket may be placed on the rim 316 of the upper body 310 of the plant hopper 308. The flat-lipped rim 348 of the piston housing 304 may then be placed on the rim 316 of the plant hopper 308 sandwiching the gasket between the rim 316 of the plant hopper and the rim 348 of the piston housing 304. A clamp 358 may then be clamped around the rim 316 of the plant hopper 308 and the rim 348 of the piston housing 304 such that a hermetic seal is created between the piston housing 304 and the plant hopper 308.

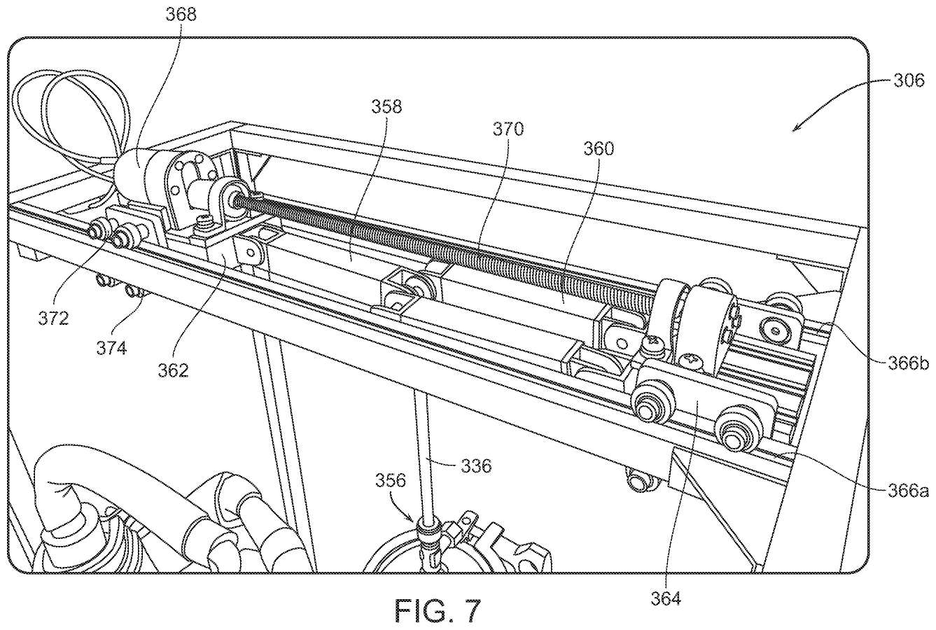

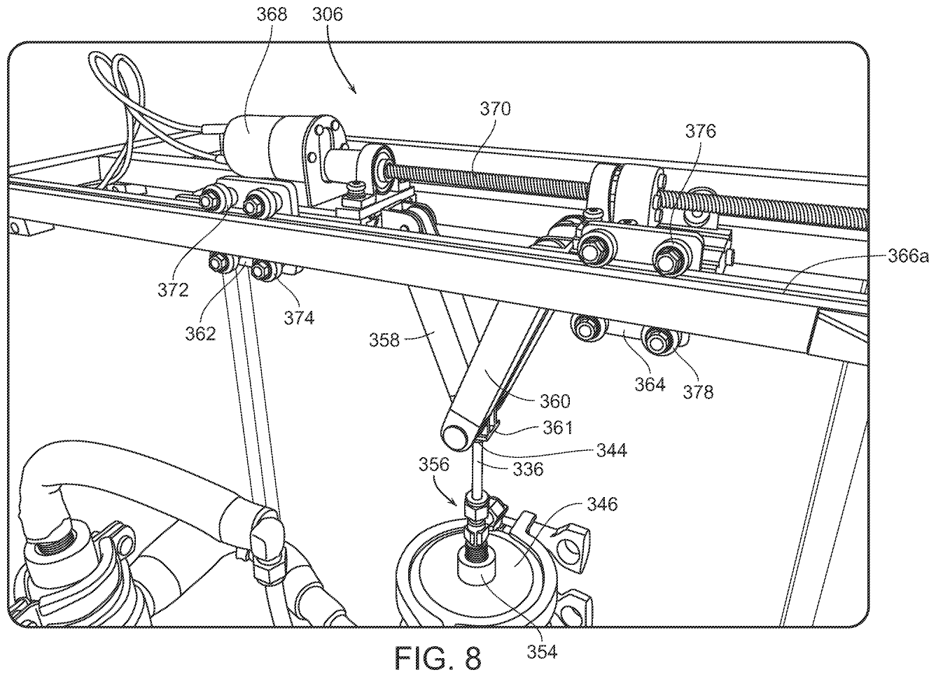

With reference now specifically to FIGS. 7 and 8, the piston drive system 306 is best illustrated. In one embodiment, the piston drive system 306 may be a mechanical scissor-type drive system. For example, piston drive system 306 may be comprised of a first arm 358 with first and second ends, a pair of second arms 360 with first and second ends, a piston rod connection 361, a first roller platform 362, a second roller platform 364, roller tracks 366, a motor 368 and a threaded bar 370. The first roller platform 362 may be comprised of one or more upper wheels 372, one or more lower wheels 374, and means to secure the motor 368. The second roller platform 364 may be comprised of one or more upper wheels 376, one or more lower wheels 378, and means to secure and receive the threaded bar 370.

The first end of the first arm 358, the piston rod connection 361, and the first ends of the pair of second arms 360 may be hingedly connected together. The second end 344 of the piston rod 336 may be connected to the piston rod connection 361. The second end of the first arm 358 may be hingedly connected to the first roller platform 362. The second ends of the pair of second arms 360 may be hingedly connected to the second roller platform 364. Thus, when the first roller platform 362 and the second roller platform 364 are moved closer together by the motor 368, the second ends of the first arm 358 and pair of second arms 360 along with the piston rod connection 361 are pushed downward pushing the piston 302 downward. Similarly, when the first roller platform 362 and the second roller platform 364 are moved further apart from each other by the motor 368, the second ends of the first arm 358 and pair of second arms 360 along with the piston rod connection 361 are pulled upward, pulling the piston 302 upward.

The one or more upper wheels 372 and 376 and the one or more lower wheels 374 and 378 of the first and second platforms 362 and 364 may sandwich the roller tracks 366. One roller track 366 may be provided in some embodiments or two roller tracks 366a and 366b may be provided wherein a roller track 366 may be placed on either side of the platforms 362 and 364 such that upper wheels 372 and 376 and lower wheels 374 and 378 of the platforms 362 and 364 sandwich both of the roller tracks 366a and 366b. The motor 368 may be comprised of means to rotate the threaded bar 370 along its axis. When the threaded bar 370 is rotated in one direction along its axis by the motor 368, the first and second roller platforms 362 and 364 are rolled closer together and thus push the piston 302 down as described above. When the threaded bar 370 is rotated in the opposite direction along its axis by the motor 368, the first and second roller platforms 362 and 364 are rolled further away from each other and thus pull the piston 302 upward as described above.

In alternative embodiments, the piston drive system 306 may be a pneumatic-type system or any other known system to move a piston up and down known in the art.

With reference now to FIGS. 2, 3, and 10, the evaporation chamber subsystem 400 is best illustrated. Generally, the evaporation chamber subsystem 400 may be comprised of a lid 402, an evaporation chamber 404, a heat source 406, and a scissor stand platform 408. Generally, the evaporation chamber subsystem 400 may function to receive the solvent and solute mixture from the plant hopper 308 and to evaporate the solvent from the solute to create a purified plant extract.

The lid 402 may be comprised of a plant hopper connection 410, a vacuum gauge 412, a sight glass 414, a thermometer 416, a vacuum seal release 418, a solvent vapor escape 420, and an insulated barrier 422. The plant hopper connection 410 may fluidly connect the plant hopper 308 to the lid 402 such that the solvent and solute solution from the plant hopper may flow through the lid and into the evaporation chamber 404. In some embodiments, the plant hopper connection 410 may be located centrally on the lid 402.

The vacuum gauge 412 may be comprised of a face and a probe. The face may be positioned above the lid 402 while the probe may extend through the lid 402 and into the evaporation chamber 404 so as to read the pressure in the evaporation chamber 404. The face of the vacuum gauge 412 may provide a reading as to the pressure inside of the evaporation chamber 404. The face may be analog or may be digital. A hermetic seal may be made between the probe and the lid 402.

The sight glass 414 may allow a user to look through the lid 402 and into the evaporation chamber 404 to view the process of evaporating the solvent. The sight glass 414 may be made of any strong and durable transparent material with a high melting point to withstand the temperature of the evaporation chamber. The sight glass 414 may be positioned such that a user of the extraction apparatus 100 may easily and conveniently peer into the evaporation chamber 404. A hermetic seal may be created between the lid 402 and the sight glass 414.

The thermometer 416 may be comprised of a face and a probe. The face may be positioned above the lid 402 while the probe may extend through the lid 402 and into the evaporation chamber 404 so as to read the temperature in the evaporation chamber 404. The face of the thermometer 416 may provide a reading as to the temperature inside of the evaporation chamber 404. The face may be analog or may be digital. A hermetic seal may be made between the probe and the lid 402.

The vacuum seal release 418 may extend out of the lid 402 and may be comprised of a release valve 424 and a deep vacuum gauge 426. The release valve 424 may release the vacuum seal from the system to return the system to atmospheric pressure while the deep vacuum gauge 426 may give a reading as to the deep pressure of the system.

The solvent vapor escape 420 may include a vapor escape line 428 extending from the lid 402 that allows solvent vapor to escape from the evaporation chamber 404 and to enter the condenser subsystem 500. A hermetic seal may be created between the solvent vapor escape 420 and the lid 402. The vapor escape line 428 may be transparent such that a user may monitor the process of solvent vapor escaping the evaporation chamber 404.

The evaporation chamber 404 may be comprised of a pot. In some embodiments, the pot may be a stainless-steel pot and may be double handled. Additionally, the pot may contain a layer of insulation wrapped around the exterior of the pot. Preferably, the pot may be comprised of a thick and flat rim so as to be able to create a hermetic seal with the lid 402. A stir rod may also be included in the evaporation chamber 404. The stir rod may be a magnetic stir rod that is activated by the heat source 406.

As stated above, when in use, the lid 402 may be hermetically sealed to the evaporation chamber 404. In some embodiments, the lid 402 may be stationarily secured to the frame 102 of the extraction apparatus 100. To create the hermetic seal between the lid 402 and the evaporation chamber 404, a gasket may be placed on the rim of the evaporation chamber 404. The evaporation chamber 404 with the gasket may be place atop the heat source 406 which may be secured to the scissor stand platform 408. The evaporation chamber 404 may then be raised to the lid 404 by the scissor stand platform 408 such that the evaporation chamber 404 is touching the lid 402 with the gasket sandwiched between the lid 402 and the evaporation chamber 404. A clamp may then be used to clamp down the lid 402 and the evaporation chamber 404 together. The clamp may be a stainless-steel tri-clamp.

The heat source 406 may be a heat plate as commonly known in the art with a heat control and a stir rod control. The heat control may be used to adjust the temperature of the heat plate. The stir rod control may be used to adjust the speed of the stir rod within the evaporation chamber 404. As described above, the heat source 406 may be attached to the top of a scissor stand platform 408. The scissor stand platform 408 may be raised or lowered by a manual knob or an electronic switch.

With reference now to FIGS. 2 and 6 the condenser subsystem 500 is best illustrated. The condenser subsystem 500 may be comprised of a vapor introduction line 502, a main body 504, a sight glass 506, a collection line 508 and an ice trap 714, the ice trap 714 may be one of the freezer components 700 and will be described in more detail below. In some embodiments, the condenser subsystem 500 may further include a light 510. The vapor introduction line 502 may collect solvent vapor coming from the solvent vapor escape 420 of the evaporation chamber subsystem 400 and introduce the vapor into the main body 504 of the condenser subsystem 500.

The main body 504 of the condenser subsystem 500 may be comprised of a tube, one or more solvent lines, and insulation. The vapor introduction line 502 may fluidly connect to the one or more solvent lines which may extend through the tube of the condenser and then fluidly connect back into a single solvent drip line having an end extending into the sight glass 506. The vapor introduction line 502 may extend into the tube of the main body 504 with a hermetic seal created between the vapor introduction line 502 and the tube of the main body 504. Similarly, the single solvent drip line may exit the tube of the main body 504 with a hermetic seal created between the single solvent drip line and the tube of the main body 504. The tube may have a condenser coolant line 730 connecting to the body of the tube and a condenser coolant return line 738 extending from the top of the tube. In this manner, coolant may be introduced to the condenser through the condenser coolant line 730 such that the tube of the main body 504 of the condenser fills up with coolant. The coolant may then surround the one or more solvent lines going through the tube and exit the condenser through the condenser coolant return line 738. The insulation may wrap tightly around the exterior of the main body 504 encasing the tube of the main body 504.

In an alternative embodiment, the main body 504 of the condenser subsystem 500 may be comprised of a tube, a solvent coil line, a coolant coil, and insulation. The solvent coil line may be a continuation of the vapor introduction line 502 within the main body 504 of the condenser 500. The solvent coil line may spiral tightly down the inside of the tube of the main body 504. The solvent coil line may end pointing downward inside the sight glass 506 such that an end of the solvent line may be visible within the sight glass 506. The tube of the main body 504 may be made from stainless steel. The coolant coil may wrap tightly around the outside of the tube of the main body 504. The coolant coil may function to cool the main body 504 to assist in condensing the solvent vapor into liquid as it travels down the solvent coil line within the main body 504. The insulation may wrap tightly around the exterior of the main body 504 encasing the tube and coolant coil of the main body 504. The sight glass 506 may be hermetically sealed to the bottom of the main body 504 and allow a user to see the condensed solvent dripping from the end of the solvent drip line within the sight glass 506. The collection line 508 may be hermetically sealed to the bottom of the sight glass 506 and collect the solvent dripping from the end of the solvent drip line within the sight glass 506. The collection line 508 may return the liquified solvent back to a solvent reservoir within the freezer as described further below.

In some embodiments a light 104 may be included as part of the condenser subsystem 500. The light 104 may illuminate the sight glass 506 such that a user may more easily view the solvent dripping therein.

The ice trap 714, as will be described in more detail below in connection with the freezer components 700, may function to capture any remaining evaporated solvent that may have escaped passed the condenser 500 and to condense the said remaining evaporated solvent.

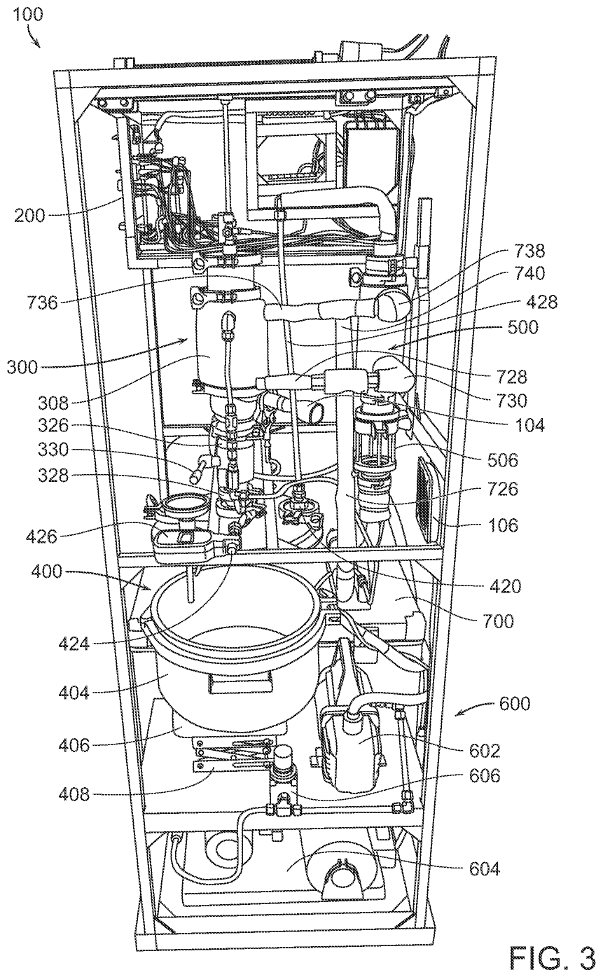

With reference now to FIG. 3, the vacuum subsystem 600 is best illustrated. The vacuum subsystem 600 may be comprised of a first vacuum pump 602, a second vacuum pump 604, a vacuum regulator 606, and various connections connecting the pumps 602 and 604 to the regulator 606 and to the internal system of the extraction apparatus 100. The vacuum subsystem 600 through the first and second vacuum pumps 602 and 604 may be used to depressurize the closed system of the extraction apparatus 100 as will be described in connection with the method below.

The first vacuum pump 602 may be a rough vacuum pump and the second vacuum pump 604 may be a deep vacuum pump. Both vacuum pumps 602 and 604 may be connected to a vacuum regulator 606 that may be adjusted by a user of the extraction apparatus 100 to introduce and to regulate a vacuum to and in the system.

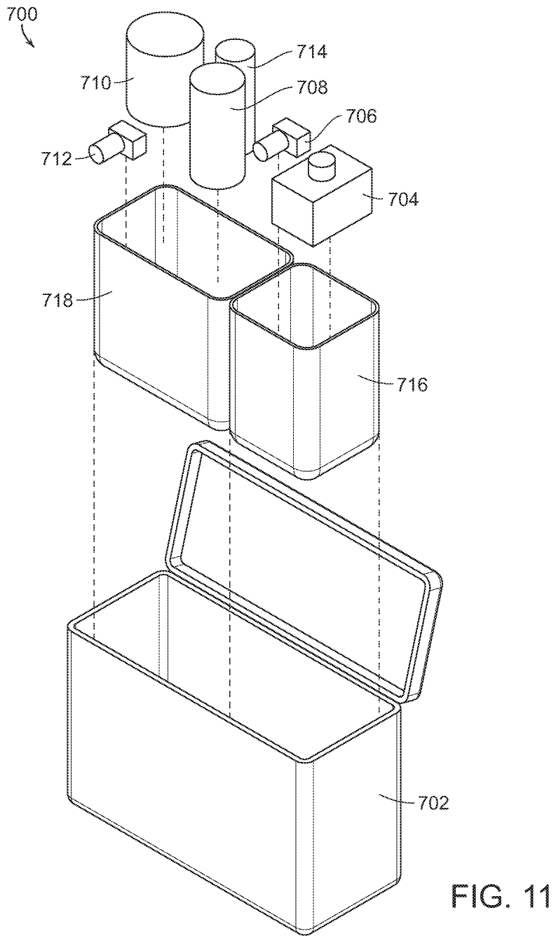

With reference now to FIGS. 11-14, the freezer components 700 are best illustrated. The freezer components 700 may include a freezer box 702, various components of a coolant subsystem which may include a coolant reservoir 704, a coolant pump 706, a thermal mass chiller 708, and various components of a solvent subsystem which may include a solvent reservoir 710, a solvent pump 712, and an ice trap 714. In some embodiments, the freezer components 700 may include dividers 716 and 718. In general, the freezer box 702 may function to cool and to maintain the temperature of the solvent and the coolant within the freezer 702.

The freezer box 702, in some embodiments, may be an electrical freezer box capable of continuously maintaining a cool temperature. Further, the freezer box 702 may be in the style of a freezer chest having a lid which opens at the top of the freezer. The freezer box 702 may also include clamps to keep the lid of the freezer box 702 clamped shut.

The coolant reservoir 704 may be designed to store, release, and collect coolant within the freezer box 702. The coolant reservoir 704 may include a coolant inlet 720, a coolant outlet 722, and a removable lid 724. The removable lid 724 may be removed to gain access to the interior of the coolant reservoir 704. With the lid 724 removed, the coolant level in the coolant reservoir 704 may be observed and coolant may be added if needed. The coolant inlet 720 may allow the coolant reservoir 704 to receive coolant that has been circulated through the extraction apparatus 100. The coolant outlet 722 may be connected to the coolant pump 706 which may pump coolant out of the coolant reservoir 704 and circulate the coolant throughout the extraction apparatus 100.

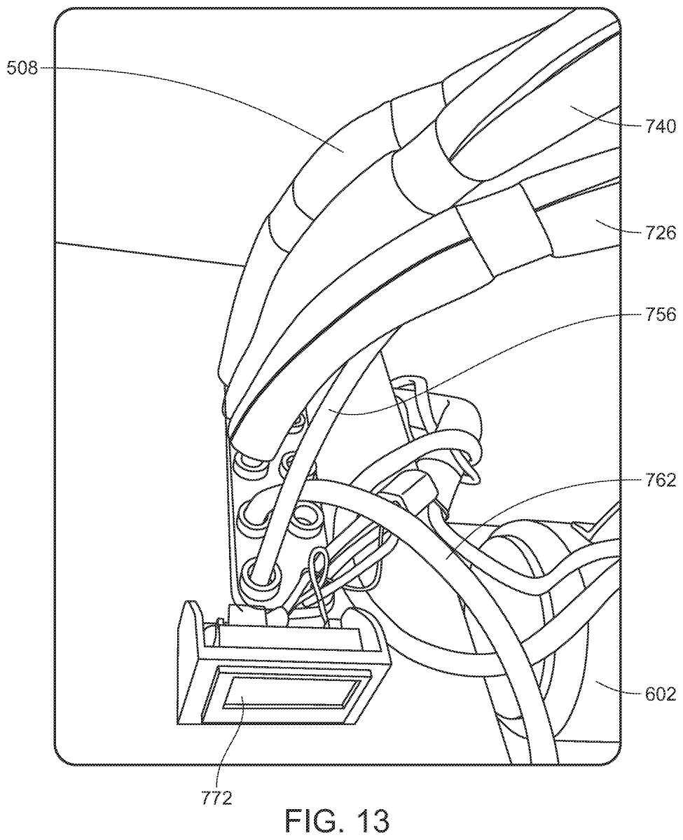

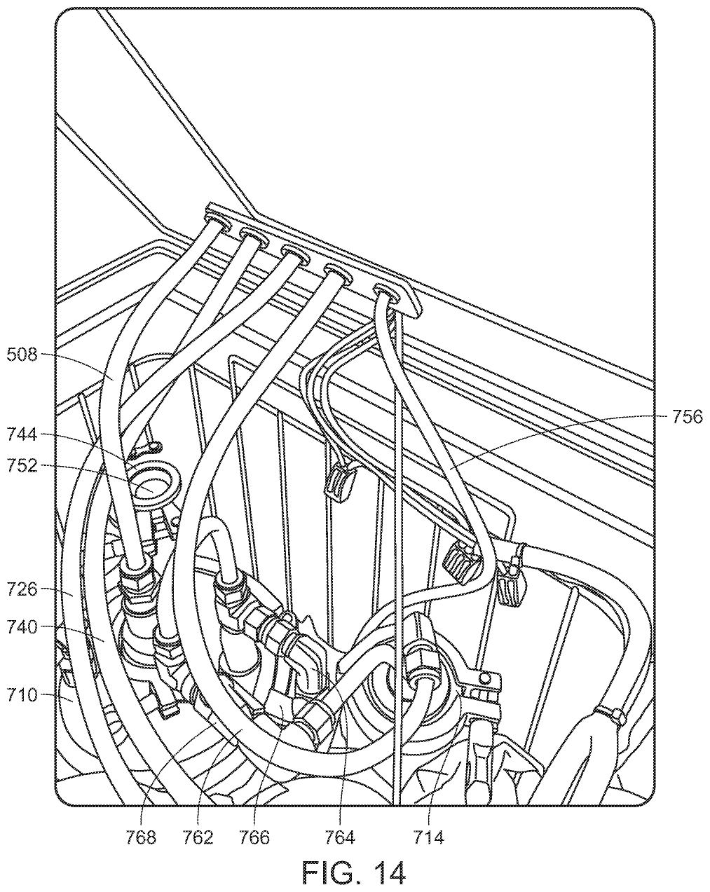

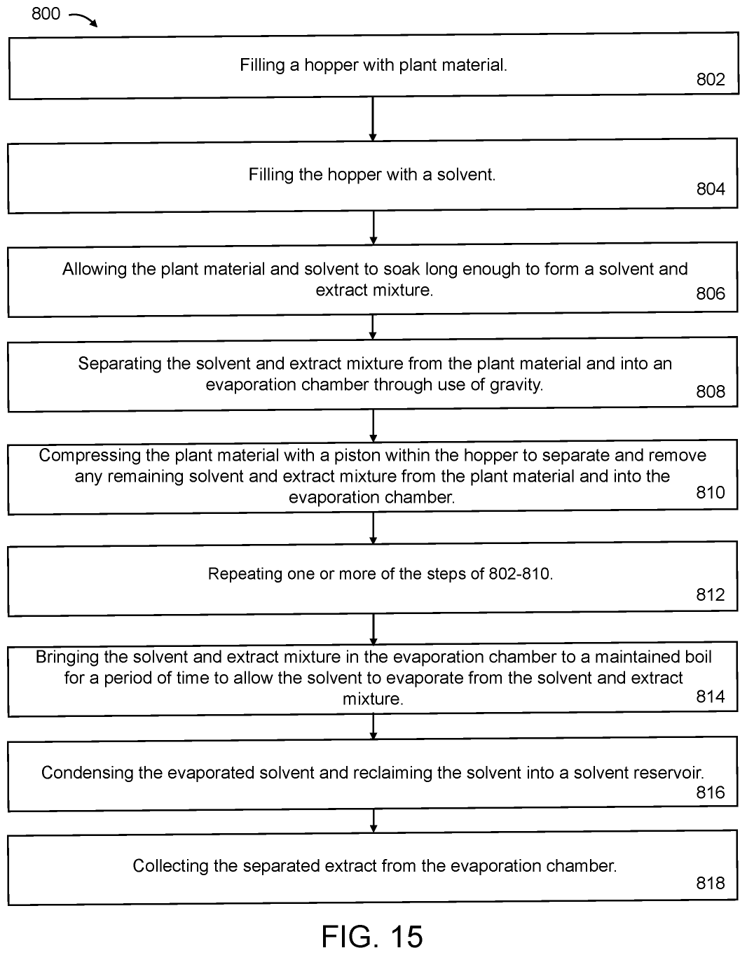

A coolant outflow line 726 may exit the coolant pump 706 and exit the freezer box 702 through the lid of the freezer box 702 (best seen in FIGS. 13 and 14). Once the coolant outflow line 726 exits the freezer box 702, it may be wrapped in an insulated material so as to keep the coolant in the coolant outflow line 726 cool. As best seen in FIGS. 3 and 4, once the coolant outflow line 726 leaves the freezer box 702, it may lead to be in between the plant hopper 308 and the main body 504 of the condenser subsystem 500.

As best seen in FIG. 6, once between the plant hopper 308 and the condenser 500, the coolant outflow line 726 may bifurcate into a plant hopper coolant line 728 and a condenser coolant line 730. At the bifurcation, the coolant outflow line 726 may contain a plant hopper valve 732 and a condenser valve 734. Both the plant hopper valve 732 and the condenser valve 734 may function to block or allow the flow of coolant to the plant hopper 308 and the condenser 500 respectively.

When the plant hopper valve 732 is opened and the condenser valve 734 is closed, the coolant may flow from the coolant outflow line 726, through the plant hopper coolant line 728, through the coolant coil of the plant hopper 308, and out of the plant hopper 308 through a plant hopper coolant return line 736. When the condenser valve 734 is opened and the plant hopper valve 732 is closed, the coolant may flow from the coolant outflow line 726, through the condenser coolant line 730, through the main body 504 of the condenser 500, and out of the main body of the condenser through a condenser coolant return line 738.

The condenser coolant return line 738 and the plant hopper coolant return line 736 may then merge into one main coolant return line 740. The main coolant return line 740 may backtrack along the coolant outflow line 726 until the coolant return line 740 makes it back to the freezer. All of the coolant lines described above may be insulated when out of the freezer to assist in keeping the coolant in the lines cool.

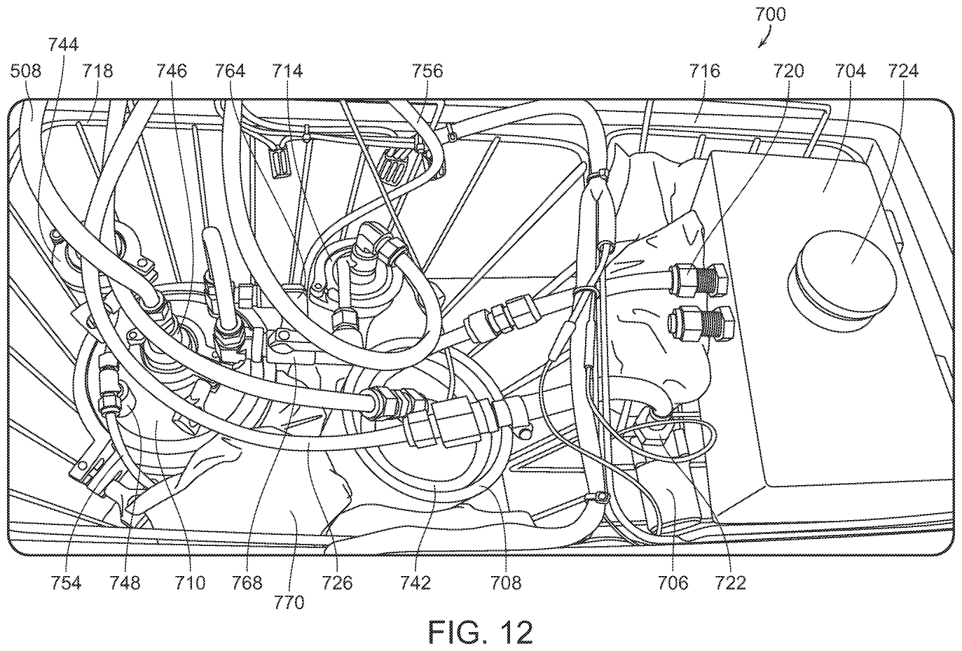

As again best seen in FIGS. 13 and 14, the main coolant return line 740 may return to the freezer box 702 through the lid of the freezer box 702. As best seen in FIG. 12, when back inside the freezer box 702, the main coolant return line 740 may enter a chiller coil 742 located in the thermal mass chiller 708, and then exit the chiller coil 742 to connect back to the coolant inlet 720 of the coolant reservoir 704. The function of the thermal mass chiller 708 is to prechill the returning coolant in the chiller coil 742 before it returns to the coolant reservoir 704. In this manner, the coolant circulates throughout the system of the extraction apparatus 100 beginning and ending at the coolant reservoir 704.

The solvent reservoir 710 may be comprised of a solvent fill 744, a solvent inlet 746, a solvent outlet 748, and a vacuum take-off line 750. The solvent reservoir may function to store, release, and collect solvent in the system of the extraction apparatus 100. In a preferred embodiment, the solvent may be heptane or hexane. In alternative embodiments, the solvent may be any solvent known in the art that is conducive for extracting oils and compounds from plant material. The solvent fill 744 may contain a sight glass 752 that allows a user of the extraction apparatus 100 to view the level of solvent in the solvent reservoir 710. The sight glass 752 may be hermetically sealed to the solvent reservoir 710 the use of a gasket and a clamp such as a stainless-steel tri-clamp. When the solvent level is low in the solvent reservoir 710, the solvent reservoir 710 may be refilled with solvent by removing the sight glass 752 and pouring solvent into the solvent fill 744. The solvent inlet 746 may receive returning solvent once it has cycled through the system of the extraction apparatus 100. The solvent outlet 748 may connect to the solvent pump 712 through a solvent outlet line 754 such that when the solvent pump 712 is turned on, it may pull solvent from the solvent reservoir through the solvent outlet 748.

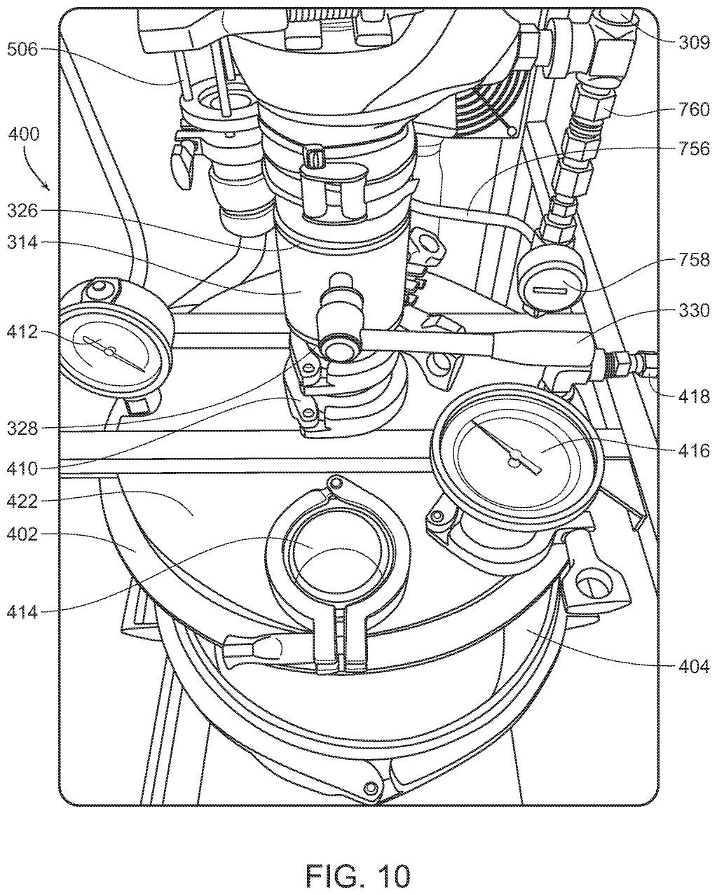

A solvent outflow line 756 may be connected to the solvent pump 712 and run from the solvent pump 712 and out of the freezer box 702 through the lid of the freezer box 702 (as best seen in FIGS. 13 and 14). Once out of the freezer box 702, the solvent outflow line 756 may run near to the valve 314 of the plant hopper 308. When the solvent outflow line 756 is near to the valve 314 of the plant hopper 308, it may connect to a solvent valve 758 (best seen in FIG. 10). When the solvent valve 758 is opened, solvent may flow into a plant hopper intake line 760. The solvent may then fill the plant hopper 308. As described above, the level of solvent in the plant hopper 308 may be indicated by the sight glass 309 of the plant hopper 308. Once in the plant hopper 308, the solvent may mix with plant material. When the valve 314 of the plant hopper 308 is opened, solvent along with any solute from the plant material may flow into the evaporation chamber 404. Once in the evaporation chamber, the solvent may be evaporated from the solute and the solvent vapor may escape through the vapor escape line 428 of the solvent vapor escape 420 (as best seen in FIG. 6). From the vapor escape line 428 the solvent vapor may enter the vapor introduction line 502 of the condenser 500. The solvent vapor will then flow through the condenser 500 where the solvent vapor will be condensed from vapor back to a liquid solvent. The solvent will then flow through the condenser 500 and drip out of the end of the solvent drip line. As described above, the drip of the solvent may be viewed through the sight glass 506 of the condenser. The solvent will drip from the end of the solvent drip line and into a solvent collection line 508. The solvent collection line 508 may then return the solvent through the lid of the freezer box 502 (as best shown in FIGS. 13 and 14) and back into the freezer box 502 where it may be returned/reclaimed to the solvent reservoir 710. In this manner, the solvent circulates throughout the system of the extraction apparatus 100 beginning and ending at the solvent reservoir 710.

The ice trap 714 may be proximate to the solvent reservoir and may include the connection for the main vacuum supply 762. Thus, a vacuum may be created by the vacuum subsystem 600 through the ice trap 714. The ice trap 714 may also include a return line 764. The ice trap 714 may function to capture any remaining solvent vapor and to condense the vapor into liquid form. The solvent liquid may then be returned to the solvent reservoir through the return line 764. The return line 764 may include a return valve 766 and a vacuum shut off valve 768. When the return valve 766 and vacuum shut of valve 768 are opened, solvent condensed in the ice trap 714 may be returned to the solvent reservoir 710 through the return line 764.

As described above, in some embodiments, a first divider 716 and a second divider 718 may be included in the freezer box 702. The first divider 716 may hold the coolant reservoir 704 and the coolant pump 706. The second divider 718 may hold the thermal mass chiller 708, the solvent reservoir 710, the solvent pump 712, and the ice trap 714. Ice blocks/bags 770 may also be included around the contents of the freezer box 702 to help maintain a cool temperature. The freezer components 700 may further include a thermometer 772 with a display that reads and displays the temperature on the inside of the freezer box 702.

With reference now to FIG. 15, a method 800 of extracting plant oil and compounds using the extraction apparatus 100 as described above, will now be disclosed. One skilled in the art would recognize that the method may include some or all of the steps described below. One skilled in the art would also recognize that the steps described below may work just as well in different order from what is described below.

One step 802 may include filling a hopper with plant material.

Another step 804 may include filling the hopper with a solvent. The solvent may be heptane or hexane.

Another method 806 may include allowing the plant material and solvent to soak long enough to form a solvent and extract mixture.

Another step 808 may include separating the solvent and extract mixture from the plant material and into an evaporation chamber through use of gravity.

Another step 810 may include compressing the plant material with a piston within the hopper to separate and remove any remaining solvent and extract mixture from the plant material and into the evaporation chamber.

Another step 812 may include repeating one or more of the steps of 802-810.

Another step 814 may include bringing the solvent and extract mixture in the evaporation chamber to a boil and maintaining the boil for a period of time to allow the solvent to evaporate from the solvent and extract mixture.

Another step 816 may include condensing the evaporated solvent and reclaiming the solvent into a solvent reservoir.

Another step 818 may include collecting the separated extract from the evaporation chamber.

Another method may include the below steps. One skilled in the art would recognize that the method may include some or all of the steps described below. One skilled in the art would also recognize that the steps described below may work just as well in different order from what is described below.

One step may be to check the liquid levels of the solvent level in the extraction apparatus 100. In this step, the solvent levels may be checked and noted. In some embodiments, the solvent level may start at 1.8 liters. It is beneficial to note the solvent level at the beginning of the method to be able to determine how much solvent was lost after the oil extraction is finished. The coolant level may also be checked to ensure enough coolant is in the system.

Another step may be to test the vacuum seal of the evaporation chamber. Before this step, the evaporation chamber should be hermetically sealed to the lid of the evaporation chamber subsystem. The valve of the plant hopper should also be closed. The vacuum seal may be tested by turning on the rough vacuum pump by flipping on the rough vacuum pump switch of the control panel. A vacuum seal is confirmed when the face of the vacuum gauge shows a vacuum created inside the evaporation chamber. Once a small amount of vacuum is presented into the evaporation chamber, the user may turn off the rough vacuum pump by flipping off the rough vacuum pump switch of the control panel and determine whether the vacuum remains constant within the evaporation chamber. If a vacuum is being created in the evaporation chamber or if the vacuum does not remain constant, the user may troubleshoot by checking the gasket and clamp that create the hermetic seal between the lid and the evaporation chamber of the evaporation chamber subsystem. Once a vacuum seal is confirmed in the evaporation chamber, the vacuum may be released from the evaporation chamber through the vacuum release in the lid of the evaporation chamber to return the chamber to atmospheric pressure. Once the vacuum is released from the evaporation chamber, the vacuum release valve may be closed.

Another step may be to chill the plant hopper. Before this step, the user may check to ensure that the plant hopper valve of the coolant line is opened, and the condenser valve of the coolant line is closed. If this is not the configuration of plant hopper valve and the condenser valve, then the user may switch the valves into this configuration. Once the valves are in the correct configuration the plant hopper may be chilled by switching the coolant pump on by turning on the coolant pump switch of the control panel. Once the coolant pump is on coolant may flow to the plant hopper. The plant hopper may be cooled to between about zero degrees Celsius and about 10 degrees Celsius. The temperature of the plant hopper may be displayed on the temperature display of the control panel.

Another step may include weighing plant material in filter paper. The plant material may be cannabis or some other plant material. The plant material may be dried prior to weighing the plant material. Once the weight of the plant material is between about 3 ounces and about 80 ounces, the plant material and filter media holding the plant material may be loaded into the plant hopper. Once the plant material and filter are loaded into the plant hopper, the piston housing may be hermetically sealed to the plant hopper as described above.

Another step may be to flood the plant hopper and its contents with solvent, which may preferably be heptane or hexane as described above. To flood the plant hopper and its contents with solvent, the solvent valve may be opened. Once the solvent valve is opened, the solvent pump may be switched on by switching on the solvent pump switch of the control panel. The solvent level in the plant hopper may be observed through the sight glass of the plant hopper after closing the solvent valve and turning off the solvent pump. If the solvent level is not near the top of the sight glass, the process may be repeated until the solvent level is observed near the top of the sight glass. If less plant material is being used, the solvent may only need to cover the plant material.

Another step may be to allow the plant material to soak in the solvent introduced into the plant hopper for between about five and about thirty minutes until a mixture of solvent and solute is obtained.

Another step may be to open the valve of the plant hopper to allow the solvent and solute mixture to flow into the evaporation chamber. This may include looking into the sight glass of the evaporation chamber to confirm that a solvent and solute mixture is flowing into the evaporation chamber.

Another step may be to lower the piston and press the plant material after the solvent and solute mixture has been drained through the valve of the plant hopper and while the valve of the plant hopper is still opened. The piston may be lowered by flipping the piston control switch of the control panel in the down direction and then using the piston speed control to push the piston down on the plant material. The piston may compress the plant material for between about one to about five minutes. After compressing the plant material, the piston may be raised by flipping the piston control switch of the control panel in the up direction and then using the piston speed control to pull the piston up.

Another step may include closing the valve of the plant hopper and filing the plant hopper with solvent for a second time similar to what was described above for the first soaking with solvent.

Another step may include soaking the plant material with the solvent in the plant hopper for a second time. The second soaking of the plant material may last about five to about fifteen minutes until a second mixture of solvent and solute is obtained.

Another step may be to open the valve of the plant hopper to allow the second solvent and solute mixture to flow into the evaporation chamber. This step may also include looking into the sight glass of the evaporation chamber to confirm that a solvent and solute mixture is flowing into the evaporation chamber.

Another step may be to turn on the stirrer of the heat source of the evaporation chamber subsystem. This may be done by switching the heat/stir switch of the control panel to an on position and turning a stir switch of the heat source to a desired stir speed.

Another step may be to stop chilling the plant hopper and to begin chilling the condenser. This may be done by closing the plant hopper valve of the coolant line and opening the condenser valve of the coolant line. This may be done with the coolant pump still turned on.

Another step may be to lower the piston a second time to press the plant material in the plant hopper in a manner similar to what was described above. This may be done with the valve of the plant hopper still open. The piston may compress the plant material for about five to about 10 minutes.

Another step may be to apply intermittent mild vacuum applications to the system while pressing the plant material for the second time with the piston. This may be done by flipping on the rough vacuum switch of the control panel and adjusting the vacuum regulator to allow a small amount of vacuum to reach the evaporation chamber. This may be monitored by the vacuum gauge of the evaporation chamber subsystem. This will allow any remaining solvent and solute solution in the plant hopper to be pulled into the evaporation chamber.

Another step may be to adjust the vacuum regulator such that the pressure in the evaporation chamber is brought down to between about five to about 15 kPa. This step may take place after the end of the second pressing the plant material with the piston.

Another step may be to close the valve of the plant hopper after the plant material is dry. This may be about one to about three minutes after adjusting the vacuum regulator so that a five to about 15 kPa reading in the evaporation chamber may be achieved. Once the valve of the plant hopper is closed, the step may include waiting for the pressure in the evaporation chamber to reach between about five to about 15 kPa.

Another step may be to raise the piston after the valve of the plant hopper is closed.

Another step may be to turn on the deep vacuum pump by flipping on the deep vacuum switch of the control panel. This may be done at about the time when the pressure in the evaporation chamber is about 10 kPa above the desired pressure. At this point, the vacuum pressure regulator may be adjusted to reach the desired pressure range.

Another step may include, once the pressure of the evaporation chamber is in the desired range, to turn off the rough vacuum pump by switching off the rough vacuum switch of the control panel.

Another step may include turning on the hot plate of the heat source after the rough vacuum pump is turned off. This may be done by adjusting the heat control on the heat source to heat up the hot plate. The temperature of the evaporation chamber may be brought to about 190 to about 210 degrees Celsius.

Another step may be to monitor and record the temperature and pressure of the evaporation chamber to ensure that a steady state distillation is achieved.

Another step may be to wait while the solvent is evaporated from the evaporation chamber through boiling the solvent and solute mixture. The solvent and solute mixture may be boiled between about 30 to about 60 minutes.

Another step may be to monitor the boiling process through the sight glass of the evaporation chamber and through the vacuum gauge and thermometer. The steady state distillation should be happening at about 10 kPa and about 30 degrees Celsius. The monitoring step may also include monitoring the sight glass of the condenser to ensure a steady drip of solvent.

Another step may be to reduce the heat of the heat source at about 15 to about 45 minutes, when the vapor temperature begins to reach about 40 degrees Celsius to about 45 degrees Celsius, and/or the condensation rate has slowed.

Another step may be to remove the heat source from the evaporation chamber. This may be done after the boiling stops and the flow of the solvent to the solvent reservoir has slowed. The heat source may be removed from the evaporation chamber by flipping off the heat/stir switch of the control panel and lowering the heat source by lowering the scissor stand platform.

Another step may be to open the system to unregulated vacuum pressure to purge the evaporation chamber from any remaining solvent. The unregulated vacuum pressure to the evaporation chamber may be held for between about 45 to about 120 minutes.

Another step may be to turn off the vacuum pumps and to return the evaporation chamber and ice trap to atmospheric pressure. This may be done by turning off the vacuum pumps and slowly releasing the vacuum through the vacuum release of the evaporation chamber until atmospheric pressure is restored to the evaporation chamber.

Another step may be to open the solvent return valve between the ice trap and the solvent reservoir to allow any solvent in the ice trap to flow back into the solvent reservoir.

Another step may be to move the scissor stand platform back up so that the evaporation chamber is supported on the heat source.

Another step may be to remove the evaporation chamber from the lid by removing the tri-clamp of the evaporation chamber and by lowering the scissor stand platform.

Another step may be to remove and store the purified plant oil from the evaporation chamber.

Another step may be to remove the spent plant material from the plant hopper.

Another step may be to weigh the spent plant material and to compare to the beginning weight of the plant material.

Another step may be to turn off the coolant pump.

Another step may be to measure and record the level of the solvent in the solvent reservoir to determine the percentage of solvent reclaimed.

Another step may be to clean the components and parts of the extraction apparatus 100 and to ensure all of the subsystems are turned off. This step may also include reassembling the oil extraction apparatus, so it is ready to go through another process.

Although specific embodiments of the extraction apparatus 100 and related method have been described in detail above for purposes of illustration, various modifications may be made without departing from the scope and spirit of the invention. Accordingly, the invention is not to be limited except as by the appended claims.

What is claimed is:

1. An extraction apparatus comprising:
a reservoir configured to hold a solvent;
a reservoir-hopper link having a first end and a second end, the first end fluidly connected to the reservoir;
a hopper configured to receive a plant material, the second end of the reservoir-hopper link being fluidly connected to the hopper and configured such that the hopper may receive solvent from the reservoir forming a plant material and solvent mixture;
a hopper-chamber link having a first end and a second end, the first end fluidly connected to the hopper;
a piston configured to be housed within the hopper and further configured to compress the plant material and solvent mixture forcing a solvent and extract mixture from the hopper;
a chamber, the second end of the hopper-chamber link being fluidly connected to the chamber, configured to receive the solvent and extract mixture from the hopper and further configured to evaporate the solvent from the solvent and extract mixture;
a chamber-condenser link having a first end and a second end, the first end fluidly connected to the chamber;
a condenser, the second end of the chamber-condenser link fluidly connected to the condenser, configured to receive the evaporated solvent from the chamber and to condense the evaporated solvent; and
a condenser-reservoir link having a first end and a second end, the first end being fluidly connected to the condenser and the second end being fluidly connected to the reservoir, configured to return the solvent from the condenser to the reservoir.

2. The extraction apparatus of claim 1, wherein the piston is removably housed within the hopper and configured to form a hermetic seal with the hopper when housed therein.

3. The extraction apparatus of claim 1, further comprising a control panel configured to dictate the operation of the extraction apparatus.

4. The extraction apparatus of claim 1, further comprising a freezer, wherein the reservoir is housed within the freezer.

5. The extraction apparatus of claim 1, further comprising an ice trap fluidly connected within the condenser-reservoir link and configured to condense any remaining evaporated solvent.

6. The extraction apparatus of claim 1, further comprising a coolant subsystem configured to lower the temperature of the hopper and condenser.

7. The extraction apparatus of claim 1, wherein the reservoir is configured to hold heptane or hexane as a solvent.

8. An extraction apparatus comprising:
a solvent subsystem comprising a solvent reservoir and a solvent reservoir pump, wherein the solvent reservoir is configured to hold a solvent and the solvent pump is configured to pump solvent out of the solvent reservoir;
a piston subsystem comprising a hopper and a piston, wherein the hopper is configured to removably house the piston, to receive a plant material, and to receive a solvent from the solvent reservoir and wherein the piston is configured to form a hermetic seal with the hopper when housed within the hopper and to compress the plant material and solvent mixture within the hopper;
an evaporation subsystem comprising a chamber having a lid and a heat source, wherein the chamber is configured to receive a solvent and extract mixture from the piston subsystem, wherein the lid is configured to removably and hermetically seal to the chamber, and wherein the heat source is configured to bring the contents of the chamber to a maintained boil to evaporate the solvent from the solvent and extract mixture;
a condenser subsystem comprising a condenser wherein the condenser is configured to receive the evaporated solvent from the evaporation subsystem and to condense the evaporated solvent and to return the condensed solvent to the solvent reservoir; and
a coolant subsystem comprising a coolant reservoir, a coolant pump, and coolant lines, wherein the coolant reservoir is configured to hold a coolant, wherein the coolant pump is configured to pump coolant from the coolant reservoir, and wherein the coolant lines are configured to circulate coolant and to cool the hopper and condenser.

9. The extraction apparatus of claim 8, further comprising a freezer housing the solvent subsystem and the coolant reservoir and coolant pump of the coolant subsystem.

10. The extraction apparatus of claim 8, further comprising a control panel configured to dictate the operation of the extraction apparatus.

11. The extraction apparatus of claim 8, further comprising a vacuum subsystem, wherein the vacuum subsystem is comprised of at least one vacuum pump, a vacuum regulator, and vacuum lines, wherein the vacuum subsystem is configured to create a vacuum within the chamber of the evaporation subsystem such that the pressure in the chamber is lower than the ambient atmospheric pressure.

12. The extraction apparatus of claim 8, wherein the evaporation subsystem is further comprised of means to stir the solvent and extract mixture.

13. The extraction apparatus of claim 8, wherein the condenser subsystem is further comprised of a sight glass configured to allow a user to determine if the condenser is functioning to condense the evaporated solvent.

14. The extraction apparatus of claim 8, wherein the solvent reservoir is configured to hold heptane or hexane as a solvent.

15. A method of extracting a plant extract comprising:
filling a hopper with plant material and a solvent;
allowing the plant material and solvent to soak to form a solvent and extract mixture;
separating the solvent and extract mixture from the plant material and into an evaporation chamber through use of gravity;
compressing the plant material with a piston within the hopper to separate any remaining solvent and extract mixture from the plant material and into the evaporation chamber;
bringing the solvent and extract mixture in the evaporation chamber to a maintained boil to separate the solvent from the extract through evaporation of the solvent;
condensing the evaporated solvent and reclaiming the solvent into a solvent reservoir; and
collecting the separated extract from the evaporation chamber.

16. The method of extracting a plant extract of claim 15, further comprising introducing a vacuum while compressing the plant material with a piston to assist in further separating any remaining solvent and extract mixture from the plant material.

17. The method of extracting a plant extract of claim 15, further comprising hermetically sealing the evaporation chamber after the chamber has received the solvent and extract mixture and introducing a vacuum to lower the pressure within the evaporation chamber;

18. The method of extracting a plant extract of claim 15, further comprising repeating the steps of allowing the plant material and solvent to soak, separating the solvent and extract mixture from the plant material through the use of gravity, and compressing the plant material with a piston to separate any remaining solvent and extract mixture from the plant material immediately after the first compression step with a piston.

19. The method of extracting a plant extract of claim 15, further comprising cooling the hopper and the condenser through the use of coolant.

20. The method of extracting a plant extract of claim 15, wherein the solvent is heptane or hexane.
